(19) United States
(12) Patent Application Publication
(10) Pub. No.: US 2019/0125231 A1
(43) Pub. Date: May 2, 2019

(54) DISPOSABLE PEN-TYPE BLOOD-RETURN-VISIBLE AND ANTI-REFLUX VENOUS BLOOD COLLECTION NEEDLE

(71) Applicant: Zhenkuan LIU, Heze City (CN)

(72) Inventor: Zhenkuan LIU, Heze City (CN)

(21) Appl. No.: 15/888,660

(22) Filed: Feb. 5, 2018

(30) Foreign Application Priority Data
Oct. 28, 2017 (CN) 201711027794.0

(51) Int. Cl. A61B 5/15; A61B 5/153

(52) U.S. Cl. CPC A61B 5/15003; A61B 5/153; A61B 5/150099; A61B 5/150343; A61B 5/150221

(57) ABSTRACT

The present invention relates to the field of medical appliances, and specifically, to a disposable pen-type blood-return-visible and anti-reflux venous blood collection needle, comprising a venous puncture needle and a transparent needle seat. A one-way valve fixation seat is arranged in a cavity of the transparent needle seat, and an anti-reflux one-way valve is mounted on the one-way valve fixation seat. The one-way valve fixation seat is plugged from a front end of the needle seat and mounted in the cavity of the needle seat. A tail end of the venous puncture needle is plugged and mounted in a blood inlet of the one-way valve fixation seat. A tail end of a puncture-end protective cover is sleeved over an outer wall of a front portion of the needle seat. A tube-plug puncture needle is plugged and mounted in a blood outlet on a tail portion of the needle seat, and a latex sheath is sleeved over the tube-plug puncture needle. An abutting-end protective cover is sleeved over the latex sheath, and a front end of the abutting-end protective cover is sleeved over an outer wall of the tail end of the puncture-end protective cover. The present invention can prevent blood in a negative-pressure blood collection container from suddenly flowing back to the human body, and can greatly reduce waste of a blood sample as the blood sample directly enters the needle seat through the venous puncture needle.

This application claims priority to Chinese application number 201711027794.0, filed 28 Oct. 2017, with a title of DISPOSABLE PEN-TYPE ANTI-REFLUX SAFE VENOUS BLOOD COLLECTION NEEDLE. The above-mentioned patent application is incorporated herein by reference in its entirety.

### TECHNICAL FIELD

The present invention relates to the field of medical appliances, and specifically, to a disposable pen-type blood-return-visible and anti-reflux venous blood collection needle.

### BACKGROUND

During venous blood collection from the human body, blood in a blood collection container often flows back to the human body. Therefore, the blood collection container is produced on a sterile basis. Currently, negative-pressure blood collection tubes are mostly made of glass. However, sterile processing in a glass blood collection container is difficult, and reagents in the blood collection container would cause fatal damage to the human body once flowing back to the human body. In addition, blood return is invisible during blood collection by using a common blood collection needle, making it difficult for medical personnel to determine whether the needle exactly punctures a blood vessel.

### SUMMARY

The objective of the present invention is to provide a disposable pen-type blood-return-visible and anti-reflux venous blood collection needle that can effectively prevent blood in a blood collection container from flowing back to the human body during venous blood collection and provide a clear view of a blood flow direction, so as to resolve the technical problem.

To achieve the objective, the present invention adopts technical solutions as follows:

A disposable pen-type blood-return-visible and anti-reflux venous blood collection needle comprises a venous puncture needle, wherein a puncture-end protective cover is sleeved over a needle body of the venous puncture needle; and further comprises a transparent needle seat, wherein a one-way valve fixation seat is arranged in a cavity of the transparent needle seat, and an anti-reflux one-way valve is mounted on the one-way valve fixation seat; the one-way valve fixation seat is plugged from a front end of the needle seat and mounted in the cavity of the needle seat; a tail end of the venous puncture needle is plugged and mounted in a blood inlet of the one-way valve fixation seat; a tail end of the puncture-end protective cover is sleeved over an outer wall of a front portion of the needle seat; a tube-plug puncture needle for connecting to a negative-pressure blood collection container is plugged and mounted in a blood outlet on a tail portion of the needle seat, and a latex sheath is sleeved over the tube-plug puncture needle; and an abutting-end protective cover is sleeved over the latex sheath, and a front end of the abutting-end protective cover is sleeved over an outer wall of the tail end of the puncture-end protective cover.

Beneficial effects: Compared with the prior art, the present invention is provided with a transparent needle seat in which an anti-reflux one-way valve is arranged. The anti-reflux one-way valve can ensure that venous blood can only flow from a venous puncture needle to an abutting tube-plug puncture needle through the transparent needle seat and the anti-reflux one-way valve. During venous blood collection from the human body, the anti-reflux one-way valve is in an open state, and after blood collection is completed, a negative-pressure blood collection container is pulled out and the anti-reflux one-way valve is closed immediately, thereby preventing blood in the negative-pressure blood collection container from suddenly flowing back to the human body. In this way, the negative-pressure blood collection container is totally isolated from the human body, that is, no damage would be caused to the human body even if the negative-pressure blood collection container is not sterile, and medical personnel can clearly see whether blood flows out during blood collection. In addition, the present invention is small-sized, making it easy to carry and conduct a blood collection operation, and waste of a blood sample can be greatly reduced as the blood sample directly enters the transparent needle seat through the venous puncture needle.

### BRIEF DESCRIPTION OF THE DRAWING

FIG. 1 is a schematic structural diagram according to an embodiment of the present invention; and

FIG. 2 is a schematic explosive structural diagram according to an embodiment of the present invention.

### DETAILED DESCRIPTION

The following describes the present invention in further detail with reference to the accompanying drawings and specific embodiments.

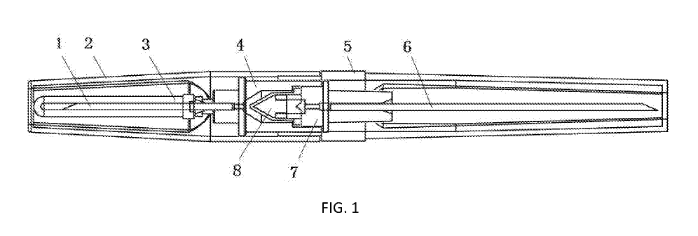

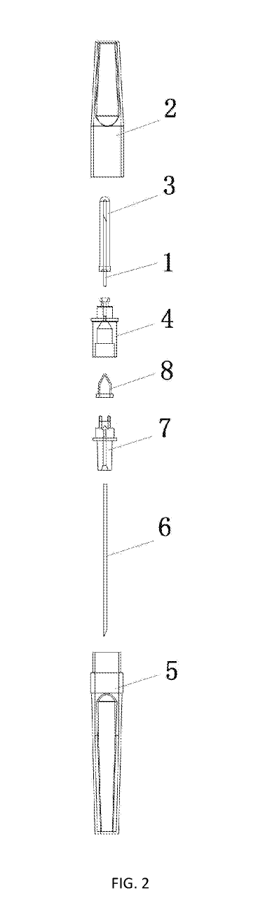

Referring to FIG. 1 and FIG. 2, the disposable pen-type blood-return-visible and anti-reflux venous blood collection needle in the present invention comprises a venous puncture needle 6 and a needle seat 4. A puncture-end protective cover 5 is sleeved over a needle body of the venous puncture needle 6. A one-way valve fixation seat 7 is arranged in a cavity of the needle seat 4, and an anti-reflux one-way valve 8 is mounted on the one-way valve fixation seat 7. The one-way valve fixation seat 7 is plugged from a front end of the needle seat 4 and mounted in the cavity of the needle seat 4. A tail end of the venous puncture needle 6 is plugged and mounted in a blood inlet of the one-way valve fixation seat 7. A tail end of the puncture-end protective cover 5 is sleeved over an outer wall of a front portion of the needle seat 4. A tube-plug puncture needle 1 for connecting to a negative-pressure blood collection container is plugged and mounted in a blood outlet on a tail portion of the needle seat 4, and a latex sheath 3 is sleeved over the tube-plug puncture needle 1. An abutting-end protective cover 2 is sleeved over the latex sheath 3, and a front end of the abutting-end protective cover 2 is sleeved over an outer wall of the tail end of the puncture-end protective cover 5.

During venous blood collection from the human body, the puncture-end protective cover 5 is taken off first, a vein is punctured by using the venous puncture needle 6, blood enters the transparent needle seat 4 through the one-way valve fixation seat 7, the anti-reflux one-way valve 8 is opened, and the blood enters the tube-plug puncture needle 1 from the blood outlet of the needle seat 4 and enters the negative-pressure blood collection container through the tube-plug puncture needle 1. After blood collection is completed, the negative-pressure blood collection container is pulled out and the anti-reflux one-way valve 8 is closed immediately, thereby preventing the blood in the negative-pressure blood collection container from suddenly flowing back to the human body. In this way, the negative-pressure blood collection container is totally isolated from the human body, that is, no damage would be caused to the human body even if the negative-pressure blood collection container is not sterile, and whether blood flows out during blood collection can be clearly seen.

The foregoing shows and describes the basic principle, main features, and technical advantages of the present invention. A person skilled in the art should understand that the described embodiments are merely for describing the principle of the present invention, various changes and improvements may be made to the present invention without departing from the spirit and scope of the present invention, and all such changes and improvements shall fall into the protection scope of the present invention. The protection scope of the present invention is subject to the appended claims and equivalents thereof.

What is claimed is:

1. A disposable pen-type blood-return-visible and anti-reflux venous blood collection needle, comprising a venous puncture needle (6), wherein a puncture-end protective cover (5) is sleeved over a needle body of the venous puncture needle (6); and further comprising a needle seat (4), wherein a one-way valve fixation seat (7) is arranged in a cavity of the needle seat (4), and an anti-reflux one-way valve (8) is mounted on the one-way valve fixation seat (7); the one-way valve fixation seat (7) is plugged from a front end of the needle seat (4) and mounted in the cavity of the needle seat (4); a tail end of the venous puncture needle (6) is plugged and mounted in a blood inlet of the one-way valve fixation seat (7); a tail end of the puncture-end protective cover (5) is sleeved over an outer wall of a front portion of the needle seat (4); a tube-plug puncture needle (1) for connecting to a negative-pressure blood collection container is plugged and mounted in a blood outlet on a tail portion of the needle seat (4), and a latex sheath (3) is sleeved over the tube-plug puncture needle (1); and an abutting-end protective cover (2) is sleeved over the latex sheath (3), and a front end of the abutting-end protective cover (2) is sleeved over an outer wall of the tail end of the puncture-end protective cover (5).
